(19) United States
(12) Patent Application Publication
(10) Pub. No.: US 2010/0256522 A1
(43) Pub. Date: Oct. 7, 2010

(54) Easily Retrieved Biological Specimen Pouch

(76) Inventors: Xing Zhou, Guangdong (CN); Li Yanfang, Guangdong (CN); Aijun Guo, Guangdong (CN)

Correspondence Address: MORGAN, LEWIS & BOCKIUS, LLP. (PA), 2 PALO ALTO SQUARE, 3000 EL CAMINO REAL, SUITE 700, PALO ALTO, CA, 94306, US

(21) Appl. No.: 12/819,950

(22) Filed: Jun. 21, 2010

Related U.S. Application Data
(63) Continuation of application No. 10/575,500, filed on Apr. 11, 2006, now abandoned.

(51) Int. Cl. A61B 10/02

(52) U.S. Cl. 600/562

(57) ABSTRACT

The invention relates to a surgical apparatus that includes an easily retrieved biological specimen pouch. The pouch has a flexible wall, a serrated open end and a closed end. There are a plurality of string-hosting slots and a plurality of notches at the open end. The plurality of string-hosting slots and the plurality of notches are alternately positioned along the open end. A control mechanism having a tube and a rod inside the tube is connected to at least one of the first and second ends of the open and retrieval string. In response to a movement of the rod relative to the tube in a first direction that pulls the open and retrieval string out of the string-hosting slots, the plurality of notches at the serrated open end are substantially drawn closer to one another, resulting in a closure of the serrated open end with a relatively small dimension.

### RELATED APPLICATIONS

This application is a continuation of U.S. patent application Ser. No. 10/575,500 filed Mar. 29, 2007, which is incorporated by reference herein in its entirety.

### TECHNICAL FIELD

This invention relates to a kind of surgical instrument used in the retrieval of biological specimens, especially a specimen pouch used in the retrieval of the removed biological specimen collected with the endoscopic equipment.

### BACKGROUND

Endoscopic surgery, celioscope surgery in particular, is a micro-invasive surgery done through a small incision. This way of surgery reduces or even eliminates the large incision so that extensive open surgery like the removal of the cholecystectomy or myoma of the uterus becomes simple clinical surgery. The recovery phase can be reduced from several weeks to several days; therefore, this technology will receive more and more extensive applications.

However, in the operation of such surgeries, where the removed biological specimen is too big to extract from the small incision, we must enlarge the incision. Thus, it greatly reduces the advantage of endoscopic surgery; another method is to divide the large biological specimen into several small tissues which are suitable for extraction from the small incision by the surgical instrument. The problem of this method is that fluid leakage or leftover tissue may remain in the body cavity, especially when the fluid-filled tissues like cholecyst, cyst, appendix inflammation or malignancy tissues are removed. In these cases, it can easily lead to infection, another syndrome or pervasion of cancer cells, endangering healthy tissues and life security.

In order to solve the difficulty of the Endoscopic surgery development, which is restricted by how to extract the large biological specimen through a small incision safely, various kinds of specimen pouches used in the retrieval of biological specimens collected with the endoscopic equipment have been developed by scientists, at home and abroad. The prior art specimen pouch is a soft pouch structure with one end open and the other closed, in which the removed biological specimen can be contained. Some examples of the specimen pouch are disclosed in U.S. Pat. No. 5,465,731 to Bell et al., U.S. Pat. No. 5,480,404 to Kammerer et al., U.S. Pat. No. 5,647,372 to Tovey et al., and U.S. Pat. No. 5,971,995 to Rousseau et al. The core of these patents is to first open the open end of the specimen pouch with an open spring, then put the removed biological specimen into the pouch. After that, by starting a retrieval switch, the open spring and the specimen pouch are separated. Then we can close the open end by a retrieval noose and take out the specimen pouch. These different patents are acquired by using different improvements to prevent the accidental separation of the open spring and specimen pouch. These products have been applied to clinical surgery effectively. What's more, in the Chinese patent No. ZL01245792.2, Jin Haiming proposed a ballonet specimen pouch. But sometimes the surgeon needs to nip the open end of the specimen pouch with the surgery instrument jaw to bring the removed specimen into the pouch; as a result, risks exist if air leaks out of the ballonet equipment. In the Chinese patent No. ZL01232360.8, Liu Fengru issued a simple-structured specimen pouch which installed a soft collecting thread at the opening of a soft plastic specimen pouch. Because no open spring or similar functional instruments are placed therein, the opening cannot automatically open. Then we need to open the pouch by using the surgery instruments under the endoscopic equipment, in order to put the removed biological specimen into the specimen pouch. In that case, the inconvenient operation of receiving the biological specimen extends the time of surgery.

It is difficult to close the prior art specimen pouch at the open end due to the rumple resulted from the large bulk of the open end and it is also hard to collect the specimen pouch through the outer sheath or small incisions. Therefore, in the Chinese patent No. ZL0215153.3, S. P. Conlon et al. designed a specimen pouch which can prevent the rumple during its retrieval and eliminate its influence when closing the pouch. Such a specimen pouch employs a flexible wall which is interspersed with soft and rigid pleats in its open end. It can produce centripetal contraction in the course of straining the retrieval noose so that it avoids the influence of the closing of the open end resulting from the anomalistic rumple. Although the influence of the closing of the open end resulting from the anomalistic rumple while straining the retrieval noose has been effectively solved in the above method, the bigger retrieval outer sheath or incisions are used to withdraw or take out the frapped open end due to the large bulk of the open end.

In order to overcome the said defects, we need to make some improvements to the prior art specimen pouch to provide one with a simpler structure, easier operation, one that is safer and more effective, especially more convenient, to relieve and collect specimens through the outer sheath. At present, there is no a specimen pouch can meet with these requirements.

### SUMMARY

The said easily retrieved biological specimen pouch comprises a flexible wall, an open end and a closed end, and the specimen pouch can receive biological specimens;

A) The flexible wall of the open end of the specimen pouch has discontinuous serration;

B) On the said serration, there are slots through which an open and retrieval string or an open spring or an retrieval noose can pass.

The said open and retrieval string can be made of the following materials: shape memory alloy wires or shape memory alloy pieces, alloy spring steel or any other materials which can save the changed shape and return to the original or near the original shape when disentangled.

The said open spring can be made of the following materials: shape memory alloy wires or shape memory alloy pieces, alloy spring steel or any other materials which can save the changed shape and return to the original or near the original shape when disentangled.

The said retrieval noose is made of the wires of macromolecule materials, compound materials or metal materials.

The said flexible wall of the specimen pouch can be made of the soft macromolecule materials or compound materials, and also the soft macromolecule materials or the compound materials enhanced by metal net or synthetic fibre, such as the following elastomer or polymer materials: Silicon Rubber, Polyurethane, Polyethylene, Polypropylene, Silicone, Ethenoid Resin or Polytetrafluoroethylene, In addition, it can be made of Silicon Rubber, Polyurethane, Polyethylene, Polypropylene, Silicone, Ethenoid Resin or Polytetrafluoroethylene, which are enhanced by memory alloy fibre net or synthetic fibre net.

The said open and retrieval string can be connected to the distant end of the inner sheath, and the specimen pouch can be installed in front of the distant end of the inner sheath and inside the distant end of the outer sheath.

One end of the said open and retrieval string can connect with a slipknot or slip block. The noose structure is formed when the other end passes through the slots in the serration of the open end of the specimen pouch and then the slipknot or slip block.

The relative position of the outer sheath and inner sheath can be fixed by an orientation button.

The said open end of the specimen pouch can be colored distinctly from the biology specimen observed under the endoscopic equipment.

According to this invention, as the open end of the specimen pouch is provided with a flexible wall of discontinuous serration, and the open and retrieval string or the retrieval noose passes through the slots on the discontinuous serration of the flexible wall of the open end of the specimen pouch, when the open and retrieval string or the retrieved noose is pulled towards the outer sheath to retrieve the specimen pouch of the invention, the bulk of contracted open end is reduced due to the discontinuous serration of the flexible wall of the open end. It is easy to close the open end of the specimen pouch, and convenient to retrieve the specimen pouch into the outer sheath or extract it through the small incision. The specimen pouch of the invention is simple, convenient, safe and efficient.

### BRIEF DESCRIPTION OF THE DRAWINGS

FIG. 1 is a perspective view illustrating the structure of the easily retrieved biological specimen pouch of this invention.

FIG. 2 is an enlarged, partially sectioned view of part A in FIG. 1.

FIG. 3 is a sectional view along line B-B in FIG. 2.

FIG. 4 is a perspective view illustrating the structure of the easily retrieved biological specimen pouch of this invention with an open and retrieval string using a slipknot noose structure.

FIG. 5 is a perspective view illustrating the structure of the easily retrieved biological specimen pouch of this invention with an open and retrieval string using a slip block noose structure.

FIG. 6 is a perspective view illustrating the structure of the easily retrieved biological specimen pouch of this invention with an open spring and a retrieval noose.

FIG. 7 is a perspective view illustrating the structure of the easily retrieved biological specimen pouch of this invention installed inside the inner sheath.

FIG. 8 illustrates the mechanism of receiving the specimen by the easily retrieved biological specimen pouch of this invention.

FIG. 9 illustrates the mechanism of collecting the easily retrieved biological specimen pouch to the outer sheath.

In the above figures, 1 denotes the specimen pouch of this invention; 1-1 denotes the flexible wall of the specimen pouch; 1-2 denotes the open end; 1-3 denotes the closed end; 1-4 denotes the serration of the flexible wall in the open end; 1-5 denotes the slot in the serration of the flexible wall of the open end; 1-6 denotes solder thread; 2 denotes the open and retrieval string; 3 denotes the open spring; 4 denotes the retrieval noose; 5 denotes the inner sheath; 5-1 denotes the distant end of the inner sheath; 5-2 denotes the near end of the inner sheath; 5-3 denotes the handle of the inner sheath; 6 denotes the outer sheath; 6-1 denotes the distant end of the outer sheath; 6-2 denotes the near end of the outer sheath; 6-3 denotes the handle of the outer sheath; 7 denotes the slipknot or slip block; 8 denotes the orientation button; 9 denotes the biological specimen.

### DESCRIPTION OF EMBODIMENTS

### Example 1

In this experiment, we first get the NiTi shape memory alloy whose recovery temperature is 30° C.˜33° C., and then make it round above its recovery temperature. Thus, the open and retrieval string 2 of this invention is formed. The open and retrieval string 2 made of the NiTi shape memory alloy can drive the expansion of the open end 1-2 of the specimen pouch; it can also function as frapping the retrieval noose and closing the open end 1-2 of the specimen pouch when collecting the pouch.

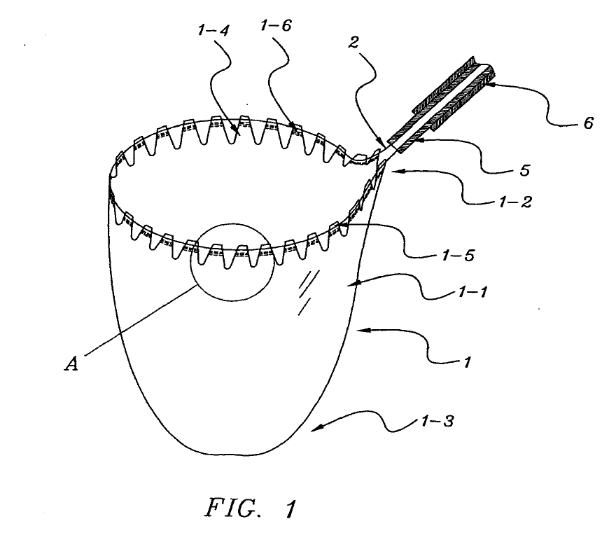

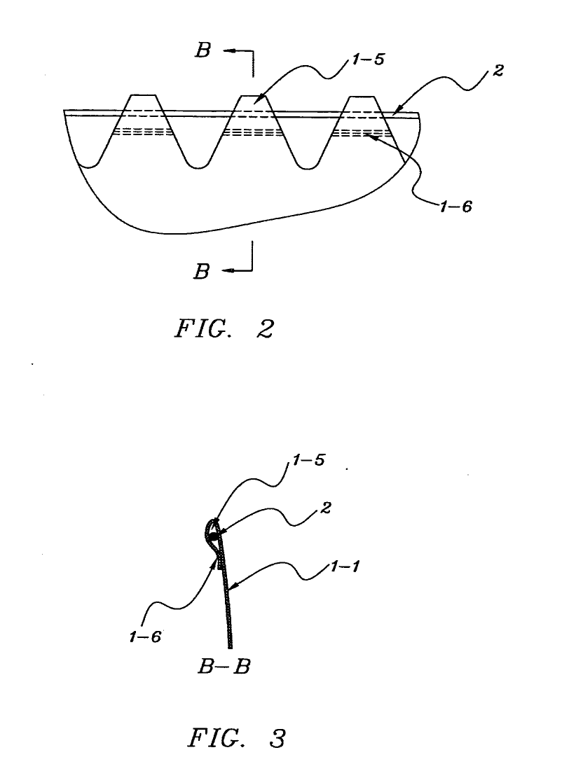

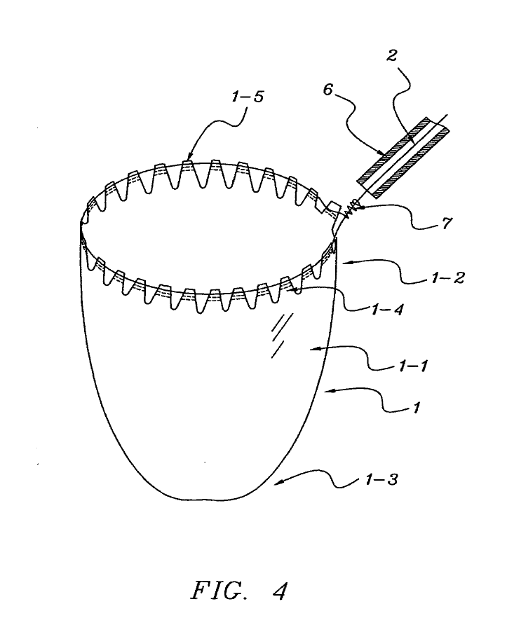

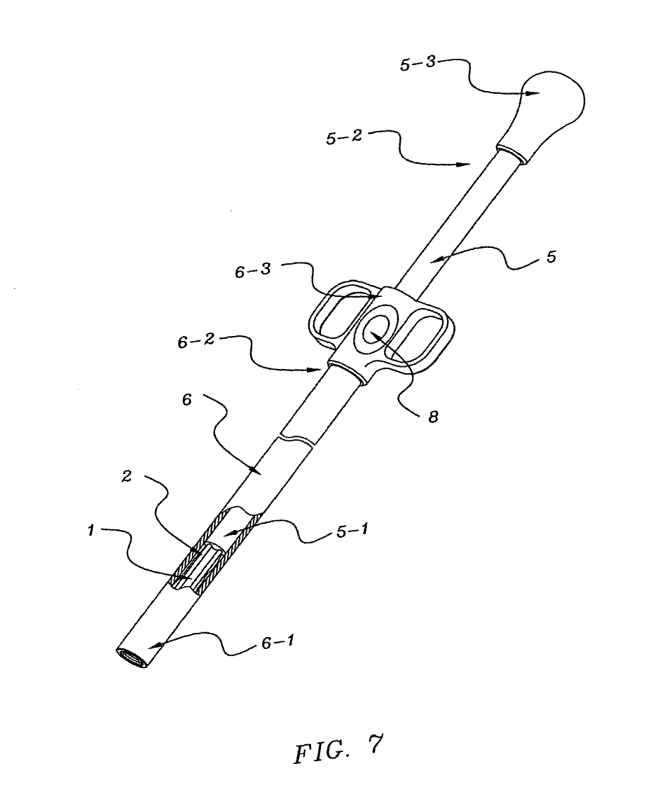

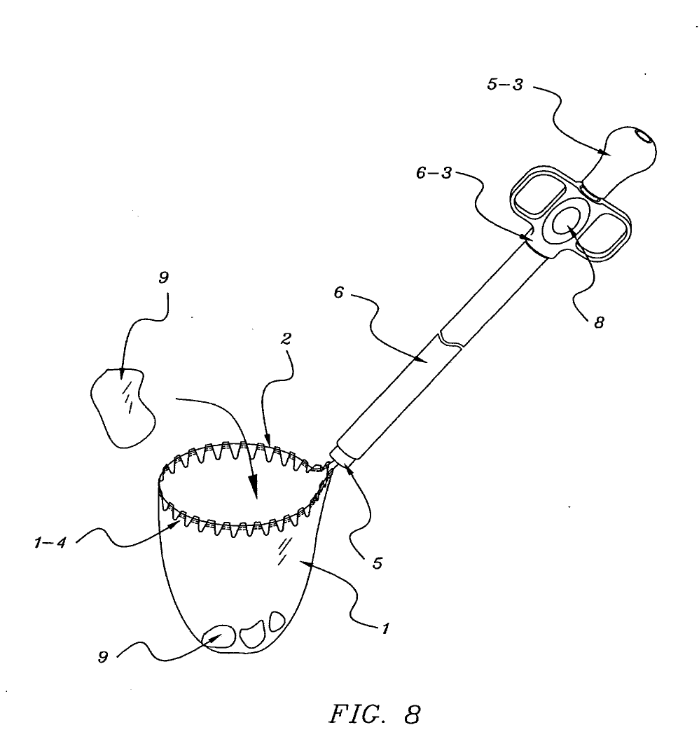

The flexible wall of the specimen pouch 1-1 is made of the transparent polyethylene membrane that forms a pocket with one closed end 1-3 and one open end 1-2 after the thermoplastic jointing. By the thermoplastic jointing, the slots 1-5 are shaped in the open end 1-2. We then cut the open end into the hackle shape 1-4, and make the open and retrieval string 2 pass through the slots 1-5 therein to connect with the distant end of the inner sheath. Below the recovery temperature, such as 15° C.˜20° C., furling the specimen pouch from the close end 1-3 to the open end 1-2, in respect that the open and retrieval string 2 is in a condition whose shape is easily modified, we can easily compress and furl it. Then we put it inside the distant end 6-1 of the outer sheath 6 and in front of the distant end 5-1 of the inner sheath 5, so the easily retrieved biological specimen pouch is obtained. FIG. 1, FIG. 2, FIG. 3 and FIG. 7 are the references.

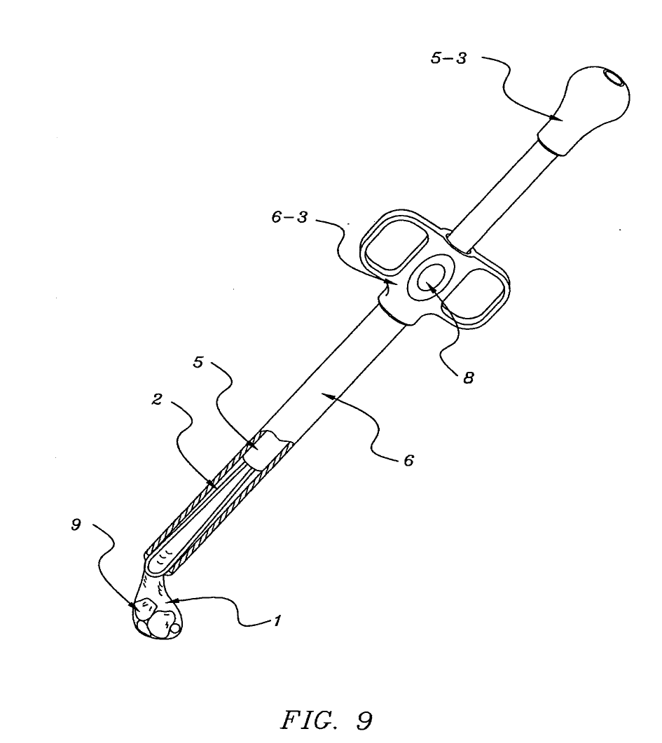

When applied in the clinical surgery, we may first insert the outer sheath into the body cavity through the small incision, and then pull forth the handle 6-3 of the outer sheath to expose the specimen pouch. With the influence of body heat, the open and retrieval string 2 recovers to the designed shape so that it drives the expansion of the open end 1-2 of the pouch 1. Therefore, the removed biological specimen 9 is brought into the specimen pouch 1 and can be extracted out of the cavity by the rinse sheath after fragmentation by the surgery instrument in the pouch 1. We may then pull forth the handle of the inner sheath 5-3 or push ahead the handle of the outer sheath 6-3, strain the open and retrieval string 2 and close the open end 1-2. Thus, the specimen pouch 1 can be collected to the outer sheath 6 and taken out of the body cavity through the small incision. FIG. 7, FIG. 8 and FIG. 9 are the references.

### Example 2

The open and retrieval string 2 is made of a super-elastic metal thread or the compound materials thread, which passes through the slots 1-5 in the serration 1-4 of the open end 1-2 in the pouch 1 and forms the slipknot structure on one end. When collecting the pouch 1 by the outer sheath 6, we can pull forth the open and retrieval string 2 through the central bore of the outer sheath 6, then strain the open end 1-2 of the pouch 1. The last step is to pull the pouch with the strained open end to the outer sheath 6. FIG. 4 is the reference.

### Example 3

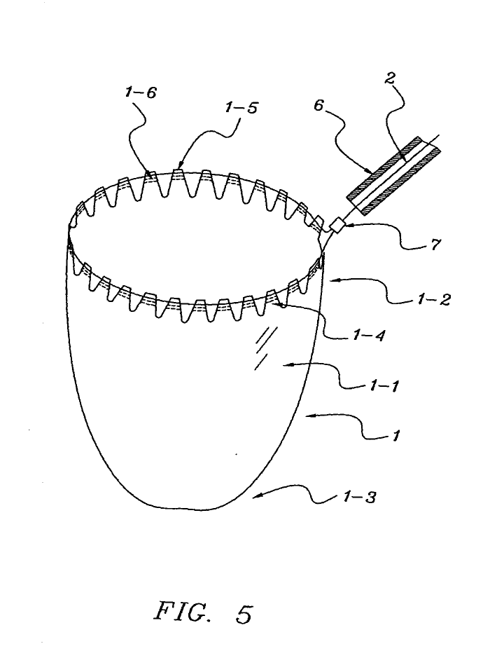

The open and retrieval string 2 is made of the NiTi memory alloy wires that are disposed with the case-hardened heat treating, while the slip block 7 is made of stainless steel. One end of the open and retrieval string 2 is fixed on the slip block 7 while the other end passes through the slots 1-5 and then through the connect bore of the slip block 7, the noose is formed hereby. When collecting the pouch 1 by the outer sheath 6, we can pull forth the open and retrieval string 2 through the central bore of the outer sheath 6, then strain the open end 1-2 of the pouch 1. The last step is to pull the pouch with the strained open end to the outer sheath 6. FIG. 5 is the reference.

### Example 4

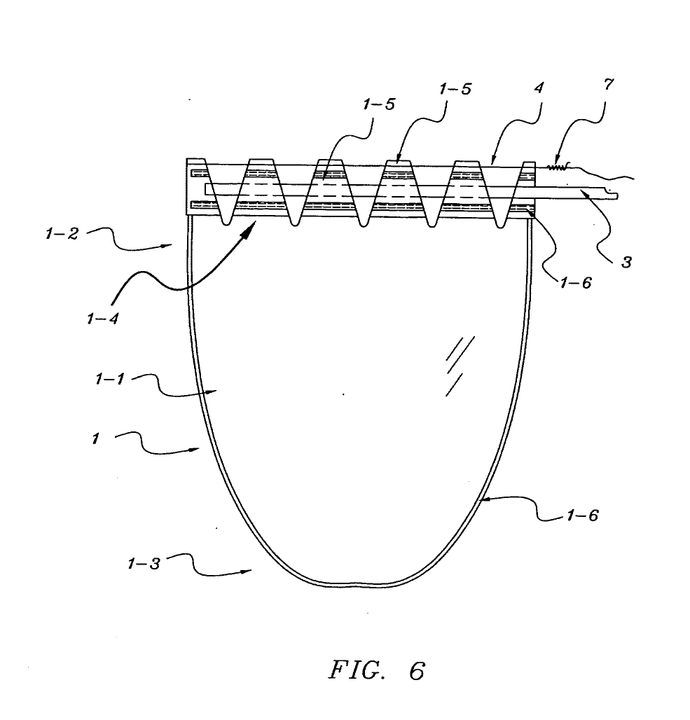

The open spring 3 and the retrieval noose 4 are employed here. After the open spring 3 expands the open end 1-2 in the pouch 1 and receives the specimen, we may withdraw it and strain the retrievable noose 4. Closing the open end 1-2 in the pouch 1, the pouch 1 can be taken out through the small incision after collecting into the outer sheath 6. FIG. 6 is the reference.

Additionally, the said flexible wall 1-1 of the specimen pouch can be made of the soft macromolecule materials or the compound materials. It can also be made of the soft macromolecule materials and the compound materials enhanced by metal net or synthetic fibre. For examples, the said flexible wall is made of the following elastomer or polymer materials: Silicon Rubber, Polyurethane, Polyethylene, Polypropylene, Silicone, Ethenoid Resin and Polytetrafluoroethylene. Moreover, it can be made of Silicon Rubber, Polyurethane, Polyethylene, Polypropylene, Silicone, Ethenoid Resin or Polytetrafluoroethylene, which are enhanced by memory alloy fibre net or synthetic fibre net.

The open and retrieval string 2 or the open spring 3 can be made of the following materials: shape memory alloy wires or pieces, alloy spring steel or any other materials which can save the changed shape and return to the original or near the original shape when disentangled.

The orientation button 8 between the inner sheath 5 and the outer sheath 6 may fix or slack the relative position of the inner sheath 5 and outer sheath 6 in the way of concave-convex assorted structure, screw tighten structure and the eccentricity annulus fixation device.

It should be noted that the structure mentioned above in this invention can be replaced by any other structure sharing the same effects, and the examples introduced in this invention are not single structures that can be performed. Although the above examples are presented in this invention, it is to be understood by those who skilled in the art that numerous variations, improvements and substitutes may be effected without departing from the invention. Therefore, it should be define the range of protection according to the spirit and scope of the claims of this invention.

What is claimed is:

1. A surgical apparatus, comprising:
a pouch having a flexible wall, a serrated open end, and a closed end, wherein there are a plurality of string-hosting slots and a plurality of notches at the serrated open end, wherein the plurality of string-hosting slots and the plurality of notches are alternately positioned along the serrated open end;
an open and retrieval string having first and second ends, wherein the first end of the open and retrieval string is configured to serially pass through the plurality of string-hosting slots at the serrated open end of the pouch; and
a control mechanism having a tube and a rod inside the tube, wherein the rod has one end that is connected to at least one of the first and second ends of the open and retrieval string,
wherein the plurality of notches at the serrated open end are substantially closed in response to a movement of the rod relative to the tube in a first direction that pulls the open and retrieval string out of the plurality of string-hosting slots.

2. The surgical apparatus of claim 1, wherein the open and retrieval string is configured to have a predefined shape when the open and retrieval string is substantially free from an external force or has a temperature of 30° C.˜33° C. or both.

3. The surgical apparatus of claim 1, wherein the open and retrieval string is made of a material selected from the group consisting of shape memory alloy and spring steel alloy.

4. The surgical apparatus of claim 1, wherein the control mechanism further includes a button for stopping the relative movement between the rod and the tube.

5. The surgical apparatus of claim 1, wherein the first end of the open and retrieval string has a noose and the second end of the open and retrieval string is configured to pass through the noose to be connected to the rod.

6. The surgical apparatus of claim 1, wherein the plurality of string-hosting slots are formed by folding a portion of the flexible wall near the serrated open end inwards with respect to the pouch so that the pouch has a smooth outer surface.

7. The surgical apparatus of claim 1, wherein there are a plurality of spring-hosting slots at the serrated open end, each spring-hosting slot being substantially parallel to a respective string-hosting slot, further comprising a pouch open spring having first and second ends, wherein the first end of the pouch open spring is configured to serially pass through the plurality of spring-hosting slots at the serrated open end of the pouch.

8. The surgical apparatus of claim 1, wherein the flexible wall near the serrated open end has a color distinct from one or more biological specimens to be collected into the pouch.

9. A surgical apparatus, comprising:
a pouch having a flexible wall, an open end, and a closed end, wherein the open end has one or more string-hosting slots;
an open and retrieval string having first and second ends, wherein the first end of the open and retrieval string is configured to serially pass through the one or more string-hosting slots, wherein the open and retrieval string is configured to automatically expand the open end of the pouch to a predefined shape when the open and retrieval string is under a predefined condition; and
a control mechanism having a tube and a rod inside the tube, wherein the rod has one end that is connected to at least one of the first and second ends of the open and retrieval string,
wherein the open and retrieval string is configured to substantially close the open end in response to a movement of the rod relative to the tube in a first direction that pulls the open and retrieval string out of the one or more string-hosting slots.

10. The surgical apparatus of claim 9, wherein the plurality of string-hosting slots are separated from one another by a plurality of notches that are alternately positioned along the open end.

11. The surgical apparatus of claim 9, wherein the predefined condition is that the open and retrieval string is substantially free from an external force or the open and retrieval string has a temperature of 30° C.˜33° C. or both.

12. The surgical apparatus of claim 9, wherein the open and retrieval string is made of a material selected from the group consisting of shape memory alloy and spring steel alloy.

13. The surgical apparatus of claim 9, wherein the control mechanism further includes a button for stopping the relative movement between the rod and the tube.

14. The surgical apparatus of claim 9, wherein the first end of the open and retrieval string has a noose and the second end of the open and retrieval string is configured to pass through the noose to be connected to the rod.

15. The surgical apparatus of claim 9, wherein the plurality of string-hosting slots are formed by folding a portion of the flexible wall near the open end inwards with respect to the pouch so that the pouch has a smooth outer surface.

16. The surgical apparatus of claim 9, wherein there are a plurality of spring-hosting slots at the open end, each spring-hosting slot being substantially parallel to a respective string-hosting slot, further comprising a pouch open spring having first and second ends, wherein the first end of the pouch open spring is configured to serially pass through the plurality of spring-hosting slots at the open end of the pouch.

17. The surgical apparatus of claim 9, wherein the flexible wall is made of a material selected from the group consisting of soft macromolecule material and soft compound material, which materials are enhanced by metal net or synthetic fiber or both.

18. A method of using a surgical apparatus to collect biological specimens inside a patient's body, wherein the surgical apparatus includes a pouch having a flexible wall, a serrated open end, and a closed end, wherein there are a plurality of string-hosting slots and a plurality of notches at the serrated open end, wherein the plurality of string-hosting slots and the plurality of notches are alternately positioned along the serrated open end, an open and retrieval string having first and second ends, wherein the first end of the open and retrieval string is configured to serially pass through the plurality of string-hosting slots at the serrated open end of the pouch, and a control mechanism having a tube and a rod inside the tube, wherein the rod has one end that is connected to at least one of the first and second ends of the open and retrieval string, comprising:
inserting at least a portion of the surgical apparatus into the patient's body through an incision on the patient's body, wherein the inserted portion includes the pouch;
expanding the serrated open end of the pouch inside the patient's body to a predefined shape by moving the rod relative to the tube in a first direction;
scraping one or more biological specimens inside the patent's body into the pouch through the expanded open end;
while the pouch is inside the patent's body, substantially closing the plurality of notches at the serrated open end by moving the rod relative to the tube in a second direction that is opposite the first direction, wherein the movement pulls the open and retrieval string out of the plurality of string-hosting slots; and
after a substantial closure of the serrated open end, pulling the surgical apparatus including the biological specimens from the patent's body through the incision.

19. The method of claim 18, wherein the open and retrieval string is configured to automatically expand the serrated open end of the pouch inside the patient's body to the predefined shape when the open and retrieval string is substantially free from an external force or the open and retrieval string has a temperature of 30° C.˜33° C. or both.

20. The method of claim 18, further comprising fragmenting the one or more biological specimens to reduce their sizes before pulling the surgical apparatus including the fragmented biological specimens from the patent's body through the incision.
